(19) United States
(12) Patent Application Publication
(10) Pub. No.: US 2024/0409993 A1
(43) Pub. Date: Dec. 12, 2024

(54) ANALOG CIRCUIT TO COMPENSATE BIAS OFFSET IN AMPLIFIER ARRAY

(71) Applicant: ILLUMINA, INC., San Diego, CA (US)

(72) Inventors: Xu LIU, San Diego, CA (US); Arvin EMADI, San Diego, CA (US)

(21) Appl. No.: 18/575,242

(22) PCT Filed: Feb. 24, 2023
(86) PCT No.: PCT/US2023/013810

§ 371 Date: Dec. 28, 2023

Related U.S. Application Data
(60) Provisional application No. 63/268,728, filed on Mar. 1, 2022.

(51) Int. Cl. C12Q 1/6869

(52) U.S. Cl. CPC C12Q 1/6869

(57) ABSTRACT

In one aspect, the disclosed technology relates to systems and methods for compensating bias offset voltages of an amplifier array when using nanopore sensors to sequence polynucleotides. In one embodiment, the disclosed system for sequencing polynucleotides includes: a nanopore for sensing a polynucleotide; an amplifier configured to measure an electrical response associated with the nanopore, the amplifier having a bias offset voltage between a first input terminal and a second input terminal; and a bias compensation circuit coupled to the nanopore and the amplifier, the bias compensation circuit configured to store a voltage potential indicative of the bias offset voltage and compensate the bias offset voltage using the voltage potential.

### CROSS-REFERENCE TO RELATED APPLICATIONS

This application claims priority to U.S. Provisional Application No. 63/268,728, filed Mar. 1, 2022, the content of which is incorporated by reference in its entirety.

### BACKGROUND

Some polynucleotide sequencing techniques involve performing a large number of controlled reactions on support surfaces or within predefined reaction chambers. The controlled reactions may then be observed or detected, and subsequent analysis may help identify properties of the polynucleotide involved in the reaction. Examples of such sequencing techniques include next-generation sequencing or massive parallel sequencing involving sequencing-by-ligation, sequencing-by-synthesis, reversible terminator chemistry, or pyrosequencing approaches.

Some polynucleotide sequencing techniques utilize a nanopore, which can provide a path for an ionic electrical current. For example, as the polynucleotide traverses through the nanopore, it influences the electrical current through the nanopore. Each passing nucleotide, or series of nucleotides, that passes through the nanopore yields a characteristic electrical current. These characteristic electrical currents of the traversing polynucleotide can be recorded to determine the sequence of the polynucleotide.

### SUMMARY

Provided in examples herein are methods for sequencing biopolymers, particularly polynucleotides, and systems and kits for performing the methods.

The systems, devices, kits, and methods disclosed herein each have several aspects, no single one of which is solely responsible for their desirable attributes. Without limiting the scope of the claims, some prominent features will now be discussed briefly. Numerous other examples are also contemplated, including examples that have fewer, additional, and/or different components, steps, features, objects, benefits, and advantages. The components, aspects, and steps may also be arranged and ordered differently. After considering this discussion, and particularly after reading the section entitled “Detailed Description,” one will understand how the features of the devices and methods disclosed herein provide advantages over other known devices and methods.

In some embodiments of a nanopore nucleic acid sequencing system, the system includes an array of nanopore unit cells/sequencing cells (or “pixels”) and uses an array of amplifiers to measure small signals (e.g., currents) in the array of nanopore unit cells. In some cases, low pixel-to-pixel variation of the voltage bias across the nanopore (or “sensor”) may be needed to ensure accurate actuation of the nucleic acid and/or measurement of the nanopore signal due to the chemistry used in the nanopore sequencing reaction. However, due to process variations in integrated circuit manufacturing, it is challenging to fabricate identical amplifiers in an integrated circuit, resulting in an undesired variation of the voltage bias across the nanopore. Therefore, a method for minimizing pixel-to-pixel voltage bias variation on a nanopore/amplifier array may be needed for efficient and accurate nanopore sequencing.

In some embodiments, disclosed is an analog mechanism and a circuit design to compensate for the variation of the voltage bias on a nanopore/amplifier array. In some embodiments, the disclosed technology comprises using an offset capacitor to measure and store the offset voltage of each pixel, wherein the offset voltage would cancel out any offset in the amplifier array.

Additional details of exemplary nanopore sequencing devices and methods of operating the devices that can be used in conjunction with the present disclosure can be found in U.S. Provisional Patent Application Nos. 63/200,868 and 63/169,041 (International Patent Application Numbers PCT/US2021/038125 and PCT/US2022/020395), the entirety of each of the disclosures is incorporated herein by reference.

In some examples, a device for sequencing polynucleotides is disclosed. The device comprises: a nanopore for sensing a polynucleotide; an amplifier configured to measure an electrical response associated with the nanopore, wherein the amplifier has a bias offset voltage between a first input terminal and a second input terminal; and a bias compensation circuit coupled to the nanopore and the amplifier. The bias compensation circuit is configured to: store a voltage potential indicative of the bias offset voltage; and compensate the bias offset voltage using the voltage potential.

In some embodiments, the bias compensation circuit comprises an offset capacitor, and wherein the offset capacitor stores the voltage potential indicative of the bias offset voltage during a first operation mode. In some examples, the first operation mode is a calibration mode of the bias compensation circuit. In some embodiments, the offset capacitor compensates the bias offset voltage using the voltage potential during a second operation mode. In some examples, the second operation mode is a measurement mode of the bias compensation circuit. In some embodiments, during the second operation mode, the amplifier measures the electrical response associated with the nanopore. In some embodiments, the nanopore has a first side and a second side, and wherein the offset capacitor has a first terminal and a second terminal, and wherein during the first operation mode: the first terminal is operably connected to the first input terminal through a switching circuit; and the second terminal is operably connected to a common electrode through the switching circuit. In some embodiments, the common electrode is tied to a ground potential during the first operation mode.

In some embodiments, the nanopore is an opening in a protein or nucleic acid structure, which is deposited in a lipid or polymer membrane. In some embodiments, the nanopore is an opening in a solid-state structure. For example, the nanopore may be a polypeptide nanopore that forms an opening in a lipid, polymer, or solid-state membrane. In some embodiments, the electrical response associated with the nanopore is an ionic current through the nanopore, and wherein the ionic current is modulated by: nucleotides in the polynucleotide near a sensing zone of the nanopore, labels on nucleotides in the polynucleotide near the sensing zone of the nanopore, nucleotides being incorporated to the polynucleotide, labels on nucleotides being incorporated to the polynucleotide, or any combination thereof. In some embodiments, the device for sequencing polynucleotides further comprises an analog-to-digital-converter, wherein an output of the amplifier is fed to the analog-to-digital converter for generating a digital signal representative of the output of the amplifier.

In some examples, a method for sequencing polynucleotides is disclosed. The method comprises: providing a polynucleotide to a sequencing cell comprising a nanopore, an amplifier for measuring an electrical response associated with the nanopore, and a bias compensation circuit between the nanopore and the amplifier; storing, by the bias compensation circuit, a voltage potential indicative of a bias offset voltage between a first input terminal and a second input terminal of the amplifier; and measuring the electrical response, wherein the bias offset voltage is compensated for in the electrical response using the voltage potential.

In some embodiments, the voltage potential is stored in an offset capacitor of the bias compensation circuit. In some embodiments, storing the voltage potential comprises: connecting a first terminal of the offset capacitor to the first input terminal of the amplifier through a switching circuit; and connecting a second terminal of the offset capacitor to a common electrode through the switching circuit. In some embodiments, the common electrode and the second input terminal of the amplifier are set to a ground potential, thereby inversely charging the offset capacitor to the voltage potential equal to a voltage at the first input terminal of the amplifier. In some embodiments, the common electrode and the second input terminal of the amplifier are set to a reference potential, thereby inversely charging the offset capacitor to the voltage potential equal to a difference between voltages at the first input terminal and the second input terminal of the amplifier.

In some embodiments, measuring the electrical response comprises: connecting a first terminal of the offset capacitor to a first side of the nanopore through a switching circuit, while a second side of the nanopore is connected to a common electrode; and connecting a second terminal of the offset capacitor to first input terminal of the amplifier through the switching circuit.

In some examples, a system for sequencing polynucleotides is disclosed. The system comprises a common cis well associated with a common cis electrode and a plurality of sequencing cells. Each of the plurality of sequencing cells comprises: (1) a trans well associated with a trans electrode; (2) a nanopore for sensing a polynucleotide, the nanopore fluidically connecting the trans well to the common cis well; (3) an amplifier configured to measure an electrical response in the nanopore, the amplifier having a bias offset voltage between a first input terminal and a second input terminal; and (4) a bias compensation circuit coupled to the nanopore and the amplifier, the bias compensation circuit configured to: store a voltage potential indicative of the input offset voltage; and compensate the input offset voltage using the voltage potential.

It is to be understood that any features of the device and/or of the array disclosed herein may be combined together in any desirable manner and/or configuration. Further, it is to be understood that any features of the method of using the device may be combined together in any desirable manner. Moreover, it is to be understood that any combination of features of this method and/or of the device and/or of the array may be used together, and/or may be combined with any of the examples disclosed herein. Still further, it is to be understood that any feature or combination of features of any of the devices and/or of the arrays and/or of any of the methods may be combined together in any desirable manner, and/or may be combined with any of the examples disclosed herein.

It should be appreciated that all combinations of the foregoing concepts and additional concepts discussed in greater detail below are contemplated as being part of the inventive subject matter disclosed herein and may be used to achieve the benefits and advantages described herein.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1A schematically illustrates an example of DNA translocation through a solid-state nanopore.

FIG. 1B schematically illustrates an example of DNA translocation through a protein nanopore.

FIG. 2 schematically illustrates an example of a nanopore array with an integrated amplifier array.

FIG. 3A illustrates a circuit schematic of an amplifier and an equivalent circuit of a nanopore sensor.

FIG. 3B illustrates a schematic of a circuit that can be used for compensating bias offsets in the amplifier array of FIG. 2 in accordance with some embodiments of the disclosed technology.

FIG. 4 depicts an example circuit schematic having a bias compensation circuit that can be used for compensating bias offsets in the amplifier array of FIG. 2 in accordance with some embodiments of the disclosed technology.

FIGS. 5A and 5B depicts illustrative operations performed by the circuit of FIG. 4 to compensate bias offsets in the amplifier array according to some embodiments of the disclosed technology.

FIG. 6 depicts an example circuit schematic having a bias compensation circuit that can be used for compensating bias offsets in the amplifier array of FIG. 2 in accordance with some embodiments of the disclosed technology.

FIGS. 7A and 7B depicts illustrative operations performed by the circuit of FIG. 6 to compensate bias offsets in the amplifier array according to some embodiments of the disclosed technology.

FIG. 8 depicts a method for compensating bias offsets in the amplifier array of FIG. 2 according to some embodiments of the disclosed technology.

### DETAILED DESCRIPTION

All patents, applications, published applications and other publications referred to herein are incorporated herein by reference to the referenced material and in their entireties. If a term or phrase is used herein in a way that is contrary to or otherwise inconsistent with a definition set forth in the patents, applications, published applications and other publications that are herein incorporated by reference, the use herein prevails over the definition that is incorporated herein by reference.

### Definitions

All technical and scientific terms used herein have the same meaning as commonly understood to one of ordinary skill in the art to which this disclosure belongs unless clearly indicated otherwise.

As used herein, the singular forms “a”, “and”, and “the” include plural referents unless the context clearly dictates otherwise. Thus, for example, reference to “a sequence” may include a plurality of such sequences, and so forth.

The terms comprising, including, containing and various forms of these terms are synonymous with each other and are meant to be equally broad. Moreover, unless explicitly stated to the contrary, examples comprising, including, or having an element or a plurality of elements having a particular property may include additional elements, whether or not the additional elements have that property.

As used herein, the term “operably connected” refers to a configuration of elements, wherein an action or reaction of one element affects another element, but in a manner that preserves each element's functionality.

As used herein, the term “membrane” refers to a non-permeable or semi-permeable barrier or other sheet that separates two liquid/gel chambers (e.g., a cis well and a fluidic cavity) which can contain the same compositions or different compositions therein. The permeability of the membrane to any given species depends upon the nature of the membrane. In some examples, the membrane may be non-permeable to ions, to electric current, and/or to fluids. For example, a lipid membrane may be impermeable to ions (i.e., does not allow any ion transport therethrough), but may be at least partially permeable to water (e.g., water diffusivity ranges from about 40 μm/s to about 100 μm/s). For another example, a synthetic/solid-state membrane, one example of which is silicon nitride, may be impermeable to ions, electric charge, and fluids (i.e., the diffusion of all of these species is zero). Any membrane may be used in accordance with the present disclosure, as long as the membrane can include a transmembrane nanoscale opening and can maintain a potential difference across the membrane. The membrane may be a monolayer or a multilayer membrane. A multilayer membrane includes two or more layers, each of which is a non-permeable or semi-permeable material.

The membrane may be formed of materials of biological or non-biological origin. A material that is of biological origin refers to material derived from or isolated from a biological environment such as an organism or cell, or a synthetically manufactured version of a biologically available structure (e.g., a biomimetic material).

An example membrane that is made from the material of biological origin includes a monolayer formed by a bolalipid. Another example membrane that is made from the material of biological origin includes a lipid bilayer. Suitable lipid bilayers include, for example, a membrane of a cell, a membrane of an organelle, a liposome, a planar lipid bilayer, and a supported lipid bilayer. A lipid bilayer can be formed, for example, from two opposing layers of phospholipids, which are arranged such that their hydrophobic tail groups face towards each other to form a hydrophobic interior, whereas the hydrophilic head groups of the lipids face outwards towards the aqueous environment on each side of the bilayer. Lipid bilayers also can be formed, for example, by a method in which a lipid monolayer is carried on an aqueous solution/air interface past either side of an aperture that is substantially perpendicular to that interface. The lipid is normally added to the surface of an aqueous electrolyte solution by first dissolving it in an organic solvent and then allowing a drop of the solvent to evaporate on the surface of the aqueous solution on either side of the aperture. Once the organic solvent has at least partially evaporated, the solution/air interfaces on either side of the aperture are physically moved up and down past the aperture until a bilayer is formed. Other suitable methods of bilayer formation include tip-dipping, painting bilayers, and patch-clamping of liposome bilayers. Any other methods for obtaining or generating lipid bilayers may also be used.

A material that is not of biological origin may also be used as the membrane. Some of these materials are solid-state materials and can form a solid-state membrane, and others of these materials can form a thin liquid film or membrane. The solid-state membrane can be a monolayer, such as a coating or film on a supporting substrate (i.e., a solid support), or a freestanding element. The solid-state membrane can also be a composite of multilayered materials in a sandwich configuration. Any material not of biological origin may be used, as long as the resulting membrane can include a transmembrane nanoscale opening and can maintain a potential difference across the membrane. The membranes may include organic materials, inorganic materials, or both. Examples of suitable solid-state materials include, for example, microelectronic materials, insulating materials (e.g., silicon nitride (Si₃N₄), aluminum oxide (Al₂O₃), hafnium oxide (HfO₂), tantalum pentoxide (Ta₂O₅), silicon oxide (SiO₂), etc.), some organic and inorganic polymers (e.g., polyamide, plastics, such as polytetrafluoroethylene (PTFE), or elastomers, such as two-component addition-cure silicone rubber), and glasses. In addition, the solid-state membrane can be made from a monolayer of graphene, which is an atomically thin sheet of carbon atoms densely packed into a two-dimensional honeycomb lattice, a multilayer of graphene, or one or more layers of graphene mixed with one or more layers of other solid-state materials. A graphene-containing solid-state membrane can include at least one graphene layer that is a graphene nanoribbon or graphene nanogap, which can be used as an electrical sensor to characterize the target polynucleotide. It is to be understood that the solid-state membrane can be made by any suitable method, for example, chemical vapor deposition (CVD). In an example, a graphene membrane can be prepared through either CVD or exfoliation from graphite. Examples of suitable thin liquid film materials that may be used include diblock copolymers or triblock copolymers, such as amphiphilic PMOXA-PDMS-PMOXA ABA triblock copolymers.

As used herein, the term “nanopore” is intended to mean a hollow structure discrete from, or defined in, and extending across the membrane. The nanopore permits ions, electric current, and/or fluids to cross from one side of the membrane to the other side of the membrane. For example, a membrane that inhibits the passage of ions or water-soluble molecules can include a nanopore structure that extends across the membrane to permit the passage (through a nanoscale opening extending through the nanopore structure) of the ions or water-soluble molecules from one side of the membrane to the other side of the membrane. The diameter of the nanoscale opening extending through the nanopore structure can vary along its length (i.e., from one side of the membrane to the other side of the membrane), but at any point is on the nanoscale (i.e., from about 1 nm to about 100 nm, or to less than 1000 nm). Examples of the nanopore include, for example, biological nanopores, solid-state nanopores, and biological and solid-state hybrid nanopores. In some embodiments, a nanopore refers to a pore having an opening with a diameter at its most narrow point of about 0.3 nm to about 2 nm. For example, a nanopore may be a solid-state nanopore, a graphene nanopore, an elastomer nanopore, or may be a naturally-occurring or recombinant protein that forms a tunnel upon insertion into a bilayer, thin film, membrane, or solid-state aperture, also referred to as a protein pore or protein nanopore herein (e.g., a transmembrane pore). If the protein inserts into the membrane, then the protein is a tunnel-forming protein.

As used herein, the term “biological nanopore” is intended to mean a nanopore whose structure portion is made from materials of biological origin. Biological origin refers to a material derived from or isolated from a biological environment such as an organism or cell, or a synthetically manufactured version of a biologically available structure. Biological nanopores include, for example, polypeptide nanopores and polynucleotide nanopores.

As used herein, the term “polypeptide nanopore” is intended to mean a protein/polypeptide that extends across the membrane, and permits ions, electric current, polymers such as DNA or peptides, or other molecules of appropriate dimension and charge, and/or fluids to flow therethrough from one side of the membrane to the other side of the membrane. A polypeptide nanopore can be a monomer, a homopolymer, or a heteropolymer. Structures of polypeptide nanopores include, for example, an α-helix bundle nanopore and a β-barrel nanopore. Example polypeptide nanopores include α-hemolysin, Mycobacterium smegmatis porin A (MspA), gramicidin A, maltoporin, OmpF, OmpC, PhoE, Tsx, F-pilus, acrolysin, etc. The protein α-hemolysin is found naturally in cell membranes, where it acts as a pore for ions or molecules to be transported in and out of cells. Mycobacterium smegmatis porin A (MspA) is a membrane porin produced by Mycobacteria, which allows hydrophilic molecules to enter the bacterium. MspA forms a tightly interconnected octamer and transmembrane beta-barrel that resembles a goblet and contains a central pore.

A polypeptide nanopore can be synthetic. A synthetic polypeptide nanopore includes a protein-like amino acid sequence that does not occur in nature. The protein-like amino acid sequence may include some of the amino acids that are known to exist but do not form the basis of proteins (i.e., non-proteinogenic amino acids). The protein-like amino acid sequence may be artificially synthesized rather than expressed in an organism and then purified/isolated.

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof such as locked nucleic acids (LNA) and peptide nucleic acids (PNA). A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA, LNA, or PNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, the term “solid-state nanopore” is intended to mean a nanopore whose structure portion is defined by a solid-state membrane and includes materials of non-biological origin (i.e., not of biological origin). A solid-state nanopore can be formed of an inorganic or organic material. Solid-state nanopores include, for example, silicon nitride nanopores, silicon dioxide nanopores, and graphene nanopores.

The nanopores disclosed herein may be hybrid nanopores. A “hybrid nanopore” refers to a nanopore including materials of both biological and non-biological origins. An example of a hybrid nanopore includes a polypeptide-solid-state hybrid nanopore and a polynucleotide-solid-state nanopore.

The application of the potential difference across a nanopore may force the translocation of a nucleic acid through the nanopore. One or more signals are generated that correspond to the translocation of the nucleotide through the nanopore. Accordingly, as a target polynucleotide, or as a mononucleotide or a probe derived from the target polynucleotide or mononucleotide, transits through the nanopore, the current across the membrane changes due to base-dependent (or probe dependent) blockage of the nanopore constriction, for example. The signal from that change in current can be measured using any of a variety of methods. Each signal is unique to the species of nucleotide(s) (or probe) in the nanopore, such that the resultant signal can be used to determine a characteristic of the polynucleotide. For example, the identity of one or more species of nucleotide(s) (or probe) that produces a characteristic signal can be determined.

As used herein, the term “nanopore sequencer” refers to any of the devices disclosed herein that can be used for nanopore sequencing. In the examples disclosed herein, during nanopore sequencing, the nanopore is immersed in examples of the electrolyte disclosed herein and a potential difference is applied across the membrane. In an example, the potential difference is an electric potential difference or an electrochemical potential difference. An electrical potential difference can be imposed across the membrane via a voltage source that injects or administers current to at least one of the ions of the electrolyte contained in the cis well or one or more of the trans wells. An electrochemical potential difference can be established by a difference in ionic composition of the cis and trans wells in combination with an electrical potential. The different ionic composition can be, for example, different ions in each well or different concentrations of the same ions in each well.

As used herein, a “nucleotide” includes a nitrogen containing heterocyclic base, a sugar, and one or more phosphate groups. Nucleotides are monomeric units of a nucleic acid sequence. Examples of nucleotides include, for example, ribonucleotides or deoxyribonucleotides. In ribonucleotides (RNA), the sugar is a ribose, and in deoxyribonucleotides (DNA), the sugar is a deoxyribose, i.e., a sugar lacking a hydroxyl group that is present at the 2′ position in ribose. The nitrogen containing heterocyclic base can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. The phosphate groups may be in the mono-, di-, or tri-phosphate form. These nucleotides are natural nucleotides, but it is to be further understood that non-natural nucleotides, modified nucleotides or analogs of the aforementioned nucleotides can also be used.

As used herein, the term “signal” is intended to mean an indicator that represents information. Signals include, for example, an electrical signal and an optical signal. The term “electrical signal” refers to an indicator of an electrical quality that represents information. The indicator can be, for example, current, voltage, tunneling, resistance, potential, voltage, conductance, or a transverse electrical effect. An “electronic current” or “electric current” refers to a flow of electric charge. In an example, an electrical signal may be an electric current passing through a nanopore, and the electric current may flow when an electric potential difference is applied across the nanopore.

As used herein, the term “bias offset voltage” and “input offset voltage” are intended to mean bias voltage difference between input terminals of an active circuitry (e.g., an amplifier) due to semiconductor process variation and/or non-ideality effect. For example, both “bias offset voltage” and “input offset voltage” of an amplifier refer to the voltage potential difference between input terminals of the amplifier. Specifically, if the amplifier is a differential amplifier, both “bias offset voltage” and “input offset voltage” refer to the voltage potential difference between two input terminals of the differential amplifier.

The aspects and examples set forth herein and recited in the claims can be understood in view of the above definitions.

### Sequencing Using a Nanopore

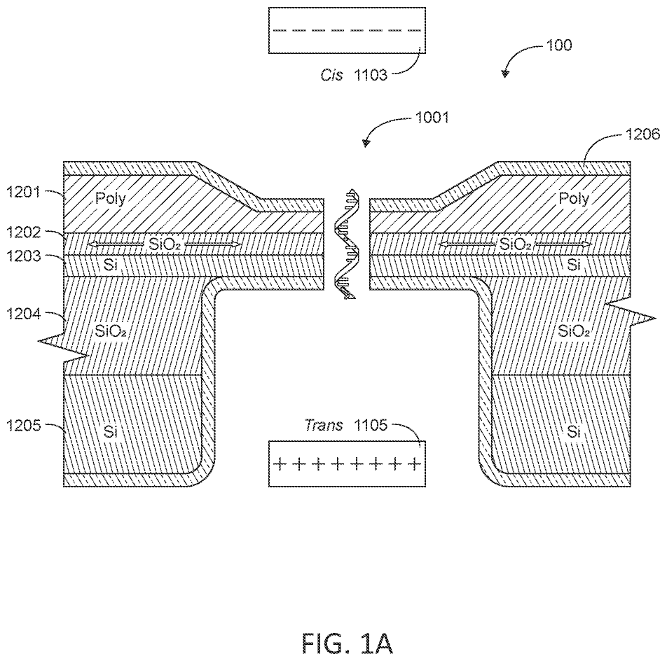

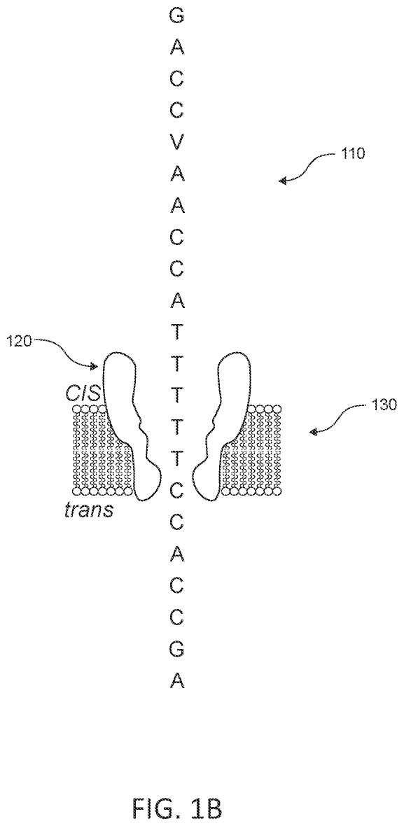

Polynucleotides may be sequenced using a nanopore unit cell, or a nanopore sensor, based on electrical responses. In some embodiments, such unit cell may include a nanopore, a flow chamber containing a liquid, one or more electrodes, and an electronic circuit for measurement. In some cases, the nanopore may be a solid-state nanopore as illustrated in FIG. 1A. In some cases, the nanopore may be a solid-state nanopore directly formed as a nanoscale opening in a membrane (e.g., silicon based, graphene, or polymer membrane). A polynucleotide may be dissolved in the liquid, e.g., an electrolyte. In some embodiments, application of a voltage via the one or more electrodes results in a driving force and/or a change in the electrical conditions that are suitable for driving translocation of the polynucleotide through the nanopore, for example from the “cis” side to the “trans” side, or vice versa. As the polynucleotide translocates through the nanopore, the polynucleotide may modulate the electrical properties of the nanopore, such that the nucleobase sequence of the polynucleotide can be identified. For example, the electrical current through the nanopore or the electrical resistance at the nanopore may be a function of the identity of the nucleobase of the polynucleotide at or near the nanopore. FIG. 1A schematically illustrates an example of a polynucleotide 1001 translocating through a solid-state nanopore device 100. The solid-state nanopore device 100 includes a silicon substrate 1205; a silicon dioxide layer 1204 formed on the silicon substrate 1205; and a stack of polysilicon 1201, silicon dioxide 1202 and silicon 1203 materials formed on the silicon dioxide layer 1204. A silicon oxide layer 1206 may be grown on the surfaces of the solid-state nanopore device 100 and may insulate the device 100. A nano-scale opening is formed in the stack of polysilicon 1201, silicon dioxide 1202 and silicon 1203 materials, allowing the polynucleotide 1001 to pass through. The solid-state nanopore device 100 may further include a cis electrode 1103 and a trans electrode 1105 for application of a voltage across the solid-state nanopore device 100. An electrolyte may be filled in the chambers between the cis electrode 1103 and the trans electrode 1105 and the silicon oxide layer 1206. The polynucleotide 1001 may be negatively charged in the electrolyte and may thus be driven through the nano-scale opening from the cis side to the trans side or vice versa when a voltage difference between the cis electrode 1103 and the trans electrode 1105 is applied.

In some cases, the nanopore may be a biological nanopore formed of peptides or polynucleotides and deposited in a lipid bilayer or a polymer membrane, e.g., a synthetic polymeric membrane. In an example shown in FIG. 1B, a protein nanopore 120 is deposited in a lipid bilayer 130. A single-stranded DNA 110 is passing, from the “cis” side, through the nanopore 120, to the “trans” side, or vice versa. Applying a voltage across the “cis” side to the “trans” side results in an ionic current through the nanopore. When a nucleotide of the DNA 110 is in or near a sensing zone of the nanopore, it may result in a unique ionic current blockade at the nanopore 120, and therefore a unique nanopore resistance depending on the identity of the nucleotide. By measuring the ionic current or the nanopore resistance, the nucleotide at or near the nanopore can be identified.

In other embodiments, the DNA 110 may not pass through the nanopore 120. A unique tag or label for a nucleotide in the DNA 110 may pass through the nanopore 120. In one example, a tag or label of the nucleotide may be a particular sequence combination of nucleotides. When the tag or label is in or near the nanopore, it may result in a unique ionic current blockade at the nanopore, and therefore a unique nanopore resistance depending on the identity of the molecule of interest. By measuring the ionic current or the nanopore resistance, the tag or label at or near the nanopore, and therefore the corresponding nucleotide, can be identified.

Although embodiments herein describe determining a signal level by determining the ionic current through the nanopore, embodiments also include, alone or in combination, determining the signal level by measuring other electrical characteristics of the cis/trans nanopore cell. For example, in some embodiments, a signal level is determined by the voltage potential at a specified area or component of the cis/trans nanopore cell. For example, in some embodiments, a signal level is determined by the electrical impedance at a specified area or component of the cis/trans nanopore cell. For example, in some embodiments, a signal level is determined by the conductivity/resistance of the nanopore membrane.

In some embodiments, sequencing of a target polynucleotide may involve nanopore sensing of (1) a single-stranded portion of the target polynucleotide; (2) a nucleic acid duplex of a portion of the target polynucleotide; (3) a label or tag that can be tethered or untethered to the target polynucleotide; or any combination thereof.

In some embodiments, multiple such nanopore unit cells may be arranged in an array, and each unit cell or each nanopore sensor may be individually accessed by a logic circuit.

### Measurement Circuit for Nanopore Sequencing

In some embodiments, a nanopore array is formed of an array of biochemical sensors, e.g., an array of nanopore unit cells described above. In some embodiments, a nanopore array can be used to perform long read DNA sequencing. A characteristic feature of a nanopore array is G-base per second per square centimeter of a chip. In some embodiments, to achieve higher data rate, the density of nanopores in a 2D array is increased. In some embodiments, a 2D readout circuit is used to take measurements from a 2D nanopore array.

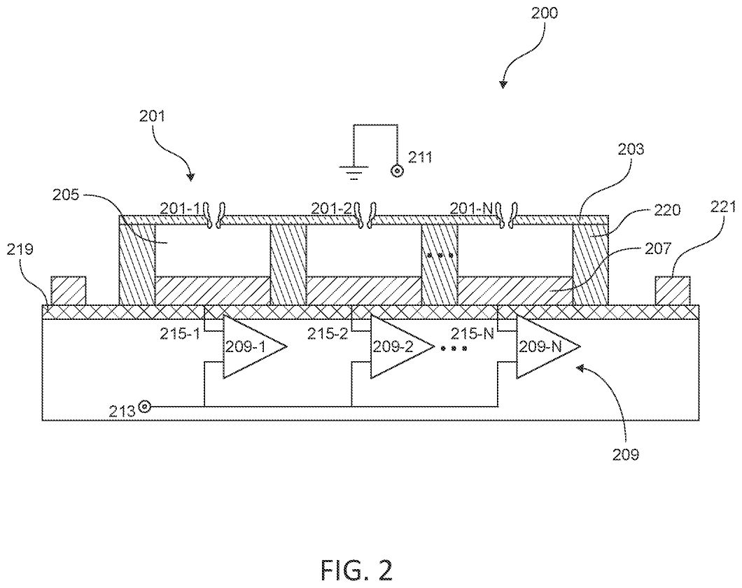

FIG. 2 schematically illustrates an embodiment of a device 200 including an amplifier array 209 integrated with a nanopore array 201. The nanopore array 201 includes the nanopore sensors 201-1, 201-2 through 201-N and the amplifier array 209 includes the amplifier 209-1, 209-2 through 209-N, with N being any positive integer number. The nanopore sensors 201-1, 201-2 through 201-N may correspond to the nanopore shown and described in conjunction with FIG. 1A or FIG. 1B. As shown in FIG. 2, the nanopore array 201 are inserted through the membrane 203 and the nanopore array 201 are electrically connected with the amplifier array 209 through the liquid conductors 205 and the metallic pads 207. Each of the nanopore sensors 201-1, 201-2 through 201-N may be electrically connected to a common electrode 211. In some embodiments, the common electrode 211 may be the cis electrode 1103 as shown in FIG. 1A. In some embodiments, the amplifier array 209 may be fabricated as part of an application specific integrated circuit (ASIC) using complementary metal-oxide semiconductor (CMOS) process. A top surface of the ASIC may be deposited with a passivation layer 219. In some examples, the nanopore array 201 may be a two-dimensional (2D) high density nanopore array. In some embodiments, a nanopore sensor (e.g., the nanopore sensor 201-1) and a corresponding liquid conductor 205 and metallic pad 207 form a sequencing cell (or unit cell) for identifying a polynucleotide passed through the nanopore sensor; and the sequencing cell can be associated with one of the amplifiers in the amplifier array 209. The sequencing cells are separated by dielectric 220. Additionally, the device 200 may further include a ground 221 distributed on the passivation layer 219.

As illustrated in FIG. 2, the nanopore sensor 201-1 is paired with the amplifier 209-1, the nanopore sensor 201-2 is paired with the amplifier 209-2, and the nanopore sensor 201-N is paired with the amplifier 209-N. In other words, there are N pairs of an amplifier and a nanopore sensor, with N being any positive integer number. As such, the amplifier 209-1 can be used to measure the electrical currents flowing through the nanopore sensor 201-1 for decoding the nucleic acid sequences passed through the nanopore sensor 201-1; the amplifier 209-2 can be used to measure the electrical currents flowing through the nanopore sensor 201-2 for decoding the nucleic acid sequences passed through the nanopore sensor 201-2; and the amplifier 209-N can be used to measure the electrical currents flowing through the nanopore sensor 201-N for decoding the nucleic acid sequences passed through the nanopore sensor 201-N.

As illustrated in FIG. 2, each amplifier of the amplifier array 209 receives two inputs. More specifically, the amplifier 209-1 receives two inputs through a first input terminal 215-1 and the common input terminal (e.g., a second input terminal) 213; the amplifier 209-2 receives two inputs through a first input terminal 215-2 and the common input terminal 213; and the amplifier 209-N receives two inputs through a first input terminal 215-N and the common input terminal 213. As such, the common input terminal 213 is connected to each of the amplifier 209-1, amplifier 209-2 through amplifier 209-N. Note that, in other embodiments, the amplifier 209-1, amplifier 209-2 through amplifier 209-N may not share a common input terminal 213. In some embodiments, the amplifier 209-1 measures the electrical current flowing through the nanopore sensor 201-1 by amplifying the difference between the voltages between the first input terminal 215-1 and the common input terminal (e.g., a second input terminal) 213.

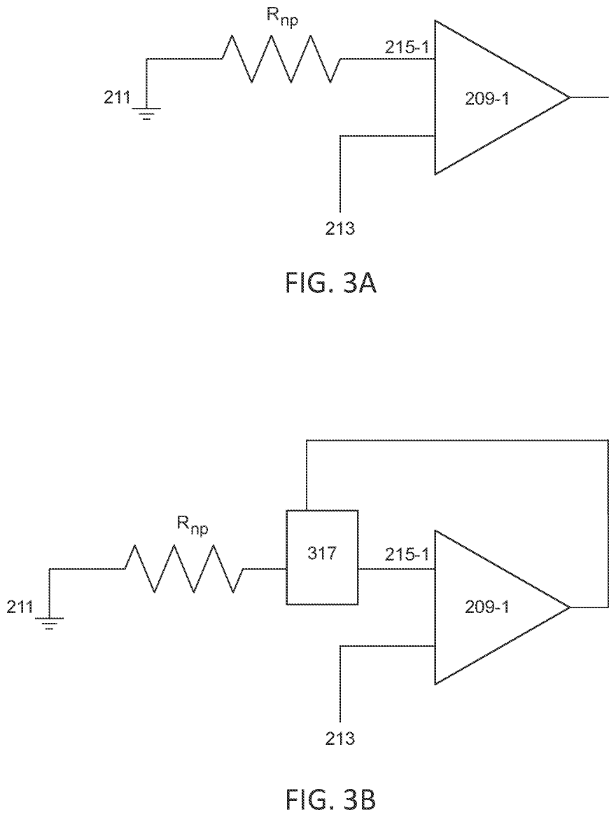

As mentioned previously, the amplifier array 209 can be used to measure electrical currents flowing through the nanopore array 201 for decoding the nucleic acid sequences. For example, when different nucleotide (e.g., the nucleotide of the DNA 110 in FIG. 1B) pass through the nanopore sensor 201-1, it may result in changes of electrical current through the nanopore sensor 201-1. Depending on the identity of the nucleotide, the nanopore sensor 201-1 may be modeled as having a corresponding resistance, i.e., the nanopore resistance Rₙₚ. The amplifier 209-1 may then sense the voltage across the nanopore resistance Rₙₚ to identify the nucleotide that is passing through the nanopore sensor 201-1. As such, the amplifier 209-1 and the equivalent circuit of the nanopore sensor 201-1 may be represented by the circuit schematic as depicted in FIG. 3A.

As shown in FIG. 3A, the amplifier 209-1 receives two inputs through the second input terminal 213 and the first input terminal 215-1. The nanopore resistance Rₙₚ is connected to the first input terminal 215-1 on one end (e.g., a first side) and is connected to the common electrode 211 on the other end (e.g., a second side). As illustrated in FIG. 3A, the common electrode 211 is biased at the ground voltage (i.e., connected to the ground). In other embodiments, though not illustrated in FIG. 3A, the common electrode 211 may be biased at any other voltages. The amplifier 209-1 may be a differential amplifier that senses the voltage difference/variation between the second input terminal 213 and the first input terminal 215-1 and magnifies that voltage difference/variation. In some applications, the amplifier 209-1 has to be able to sense small (e.g., in the order of mV) voltage variations in the second input terminal 213 and/or the first input terminal 215-1.

However, due to process variations and limitations, the amplifier array 209 may not be fabricated ideally. One non-ideality effect associated with the amplifier array 209 is the presence of bias offset between the two inputs for each of the amplifier 209-1, 209-2 through 209-N. For example, referring to FIG. 3A, there may be a constant bias offset (or bias offset voltage) between the second input terminal 213 and the first input terminal 215-1 of the amplifier 209-1 due to non-ideality effect incurred during the manufacturing process. For example, the second input terminal 213 may be biased at Vin and the first input terminal 215-1 may be biased at Vᵢₙ+V_{offset}. As a result, the voltage across the nanopore resistance Rₙₚ may be dependent upon V_{offset}. The presence of the offset voltage V_{offset} may result in poor measurement for nanopore sequencing. As such, a circuit design that compensates this voltage offset is desired.

FIG. 3B illustrates a circuit schematic including an amplifier 209-1, a nanopore resistance Rₙₚ and a bias compensation circuit 317 that compensates the bias offset of the amplifier 209-1. As shown in FIG. 3B, the bias compensation circuit 317 is coupled to the nanopore resistance Rₙₚ (the equivalent circuit of the nanopore sensor 201-1) and the amplifier 209-1. In some examples, the amplifier 209-1 may be the amplifier 209-1 of FIG. 2 and the nanopore resistance Rₙₚ may model the effective resistance of the nanopore sensor 201-1 of FIG. 2. In other examples, the amplifier 209-1 and the nanopore resistance Rₙₚ as shown in FIG. 3B may correspond to other amplifier and nanopore sensor pairs as shown in FIG. 2.

As illustrated in FIG. 3B, the common electrode 211 is biased at the ground voltage (i.e., connected to the ground). In other examples, though not illustrated in FIG. 3B, the common electrode 211 may be biased at any other voltages.

In some embodiments, the bias compensation circuit 317 may include an analog mechanism to compensate the bias offset voltage between the second input terminal 213 and the first input terminal 215-1 of the amplifier 209-1. The bias compensation circuit 317 may include an offset capacitor to store the bias offset of the amplifier 209-1. In some embodiments, the bias compensation circuit 317 may have a calibration mode and a measurement mode. For example, in the calibration mode, the offset capacitor may inversely connect to the first input terminal 215-1 of the amplifier 209-1. By setting the voltage of the second input terminal 213 of the amplifier 209-1 to 0V, the bias offset between the second input terminal 213 and the first input terminal 215-1 is stored in the offset capacitor. Subsequently, in the measurement mode, the offset capacitor and the nanopore resistance Rₙₚ are connected to the second input terminal 213 of the amplifier 209-1. The bias offset of the amplifier 209-1 then is canceled out by the voltage potential (equals to the additive inverse of the bias offset) stored in the offset capacitor. Thus, the bias voltage on the nanopore resistance Rₙₚ can be the same as the voltage of the second input terminal 213 of the amplifier 209-1 and would be independent of the bias offset V_{offset}. The operation of the bias compensation circuit 317 will be better understood by referring to detailed embodiments of the bias compensation circuit 317 as described below.

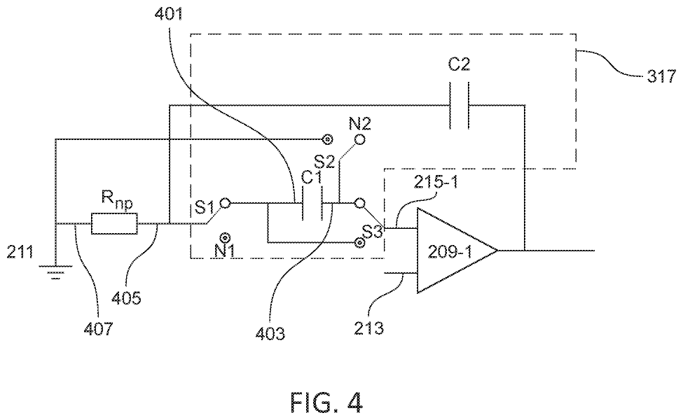

FIG. 4 illustrates an example circuit schematic of the bias compensation circuit 317 of FIG. 3B that is coupled to an amplifier 209-1 and a nanopore (represented by a nanopore resistance Rₙₚ). In some examples, the amplifier 209-1 of FIG. 4 may be the amplifier 209-1 of FIG. 2 and the nanopore resistance Rₙₚ of FIG. 4 may represent the equivalent circuit of the nanopore sensor 201-1 of FIG. 2. In other examples, the amplifier 209-1 and the nanopore resistance Rₙₚ as shown in FIG. 4 may correspond to other amplifier and nanopore sensor pairs as shown in FIG. 2.

As shown in FIG. 4, the amplifier 209-1 receives two inputs through a second input terminal 213 and a first input terminal 215-1. The nanopore resistance Rₙₚ is connected to the common electrode 211 on one end 407 (i.e., a second side of the nanopore resistance Rₙₚ). The common electrode 211 may be the cis electrode 1103 as shown in FIG. 1A. As illustrated in FIG. 4, the voltage applied to the common electrode 211 is set to 0V (i.e., ground voltage). The bias compensation circuit 317 illustratively includes an offset capacitor C1, an integration capacitor C2, a first switch S1, a second switch S2, and a third switch S3. The first switch S1, the second switch S2 and the third switch S3 selectively connect two terminals (i.e., the first terminal 401 and the second terminal 403) of the offset capacitor C1 to different nodes in the circuit schematic. Illustratively, the first switch S1 can selectively connect one end 405 (i.e., a first side) of the nanopore resistance Rₙₚ to the first terminal 401 of the offset capacitor C1 or to a floating node N1; the second switch S2 can selectively connect the second terminal 403 of the offset capacitor C1 to the common electrode 211 or to a floating node N2; and the third switch S3 can selectively connect the first input terminal 215-1 of the amplifier 209-1 to the second terminal 403 of the offset capacitor C1 or to the first terminal 401 of the offset capacitor C1. As mentioned previously, the bias compensation circuit 317 may utilize a calibration mode and a measurement mode to cancel out the bias offset of the amplifier 209-1.

In some embodiments, during the calibration mode, the first switch S1 is connected to the floating node N1, the second switch S2 connects the second terminal 403 of the offset capacitor C1 to the common electrode 211, and the third switch S3 connects the first terminal 401 of the offset capacitor C1 to the first input terminal 215-1 of the amplifier 209-1. During the measurement mode, the first terminal 401 of the offset capacitor C1 is connected to a first side 405 of the nanopore (represented by a nanopore resistance Rₙₚ) through the first switch S1; the second terminal 403 of the offset capacitor C1 is connected to the first input terminal 215-1 of the amplifier 209-1 through the third switch S3; and the second switch S2 is connected to the floating node N2. The second side 407 of the nanopore resistance Rₙₚ is connected to the common electrode 211.

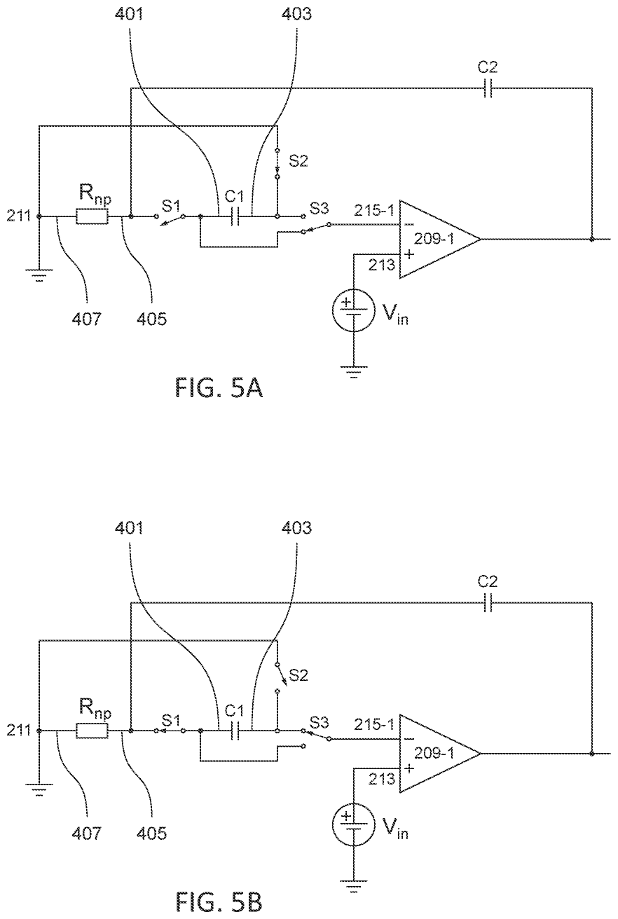

FIG. 5A illustrates an example circuit configuration when the bias compensation circuit 317 of FIG. 4 operates in a calibration mode according to some embodiments of the disclosed technology. As described above, the example circuit configuration of FIG. 5A can be obtained by connecting the first switch S1 to the floating node N1, connecting the second terminal 403 of the offset capacitor C1 to the common electrode 211 through the second switch S2, and connecting the first terminal 401 of the offset capacitor C1 to the first input terminal 215-1 of the amplifier 209-1 through the third switch S3. As shown in FIG. 5A, the voltage at the second input terminal 213 is biased at Vᵢₙ. With the presence of bias offset of the amplifier 209-1, the voltage at the first input terminal 215-1 is Vᵢₙ+V_{offset}. During the calibration mode, the offset capacitor C1 is inversely connected. In other words, the second terminal 403 of the offset capacitor C1 is connected to the common electrode 211, which is tied to a ground potential; and the first terminal 401 of the offset capacitor C1 is connected to the first input terminal 215-1 of the amplifier 209-1. In some examples, by setting Vᵢₙ to 0 voltage (i.e., tied the second input terminal 213 to the ground potential) during the calibration mode, the offset capacitor C1 is inversely charged to a voltage level-V_{offset}.

FIG. 5B illustrates an example circuit configuration when the bias compensation circuit 317 of FIG. 4 operates in a measurement mode according to some embodiments of the disclosed technology. As described above, the example circuit configuration of FIG. 5B can be obtained by connecting the first terminal 401 of the offset capacitor C1 to the first side 405 of the nanopore resistance Rₙₚ through the first switch S1, connecting the second terminal 403 of the offset capacitor C1 to the first input terminal 215-1 of the amplifier 209-1 through the third switch S3, and connecting the second switch S2 to the floating node N2. As shown in FIG. 5B, the voltage at the second input terminal 213 of the amplifier is set as Vᵢₙ. As such, the voltage at the first input terminal 215-1 remains Vᵢₙ+V_{offset}. The first terminal 401 of the offset capacitor C1 is connected to the first side 405 of the nanopore resistance Rₙₚ and the second terminal 403 of the offset capacitor C1 is connected to the first input terminal 215-1 of the amplifier 209-1. With the voltage level across the nanopore resistance Rₙₚ being the voltage level at the first input terminal 215-1 (i.e., Vᵢₙ+V_{offset}) plus the voltage level across the offset capacitor C1 (i.e., −V_{offset}), the voltage level across the nanopore resistance Rₙₚ becomes Vᵢₙ+V_{offset}−V_{offset}, which equals Vᵢₙ. As a result, the voltage across the nanopore resistance Rₙₚ can be independent of the bias offset of the amplifier 209-1. Advantageously, the cancellation of the bias offset may lead to better measurement for nanopore sequence, in contrast to the scenario illustrated in FIG. 3A.

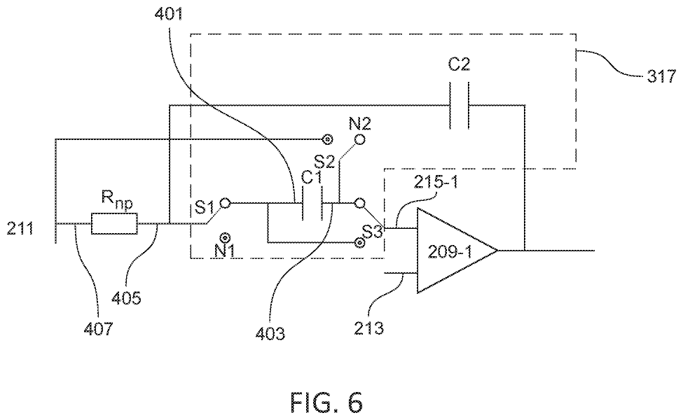

FIG. 6 illustrates an example circuit schematic of the bias compensation circuit 317 of FIG. 3B along with an amplifier 209-1 and a nanopore resistance Rₙₚ. In some examples, the amplifier 209-1 of FIG. 6 may be the amplifier 209-1 of FIG. 2 and the nanopore resistance Rₙₚ of FIG. 6 may represent the equivalent circuit of the nanopore sensor 201-1 of FIG. 2. In other examples, the amplifier 209-1 and the nanopore resistance Rₙₚ as shown in FIG. 6 may correspond to other amplifier and nanopore sensor pairs as shown in FIG. 2.

As shown in FIG. 6, the amplifier 209-1 receives two inputs through a second input terminal 213 and a first input terminal 215-1. The bias compensation circuit 317 illustratively includes an offset capacitor C1, an integration capacitor C2, a first switch S1, a second switch S2, and a third switch S3. The first switch S1, the second switch S2 and the third switch S3 selectively connect two terminals (i.e., the first terminal 401 and the second terminal 403) of the offset capacitor C1 to different nodes in the circuit schematic. Illustratively, the first switch S1 can selectively connect one end (e.g., a first side) 405 of the nanopore resistance Rₙₚ to the first terminal 401 of the offset capacitor C1 or to a floating node N1; the second switch S2 can selectively connect the second terminal 403 of the offset capacitor C1 to the common electrode 211 or to a floating node N2; and the third switch S3 can selectively connect the first input terminal 215-1 of the amplifier 209-1 to the second terminal 403 of the offset capacitor C1 or to the first terminal 401 of the offset capacitor C1. As shown in FIG. 6, the common electrode 211 is not biased at the ground voltage; instead, the common electrode will be biased at different voltages as described below.

As mentioned previously, the bias compensation circuit 317 may utilize a calibration mode and a measurement mode to cancel out the bias offset of the amplifier 209-1. In some examples, during the calibration mode, the first switch S1 is connected to the floating node N1, the second switch S2 connects the second terminal 403 of the offset capacitor C1 to the common electrode 211, and the third switch S3 connects the first terminal 401 of the offset capacitor C1 to the first input terminal 215-1 of the amplifier 209-1. During the measurement mode, the first terminal 401 of the offset capacitor C1 is connected to the first side 405 of the nanopore resistance Rₙₚ through the first switch S1; the second terminal 403 of the offset capacitor C1 is connected to the first input terminal 215-1 of the amplifier 209-1 through the third switch S3; and the second switch S2 is connected to the floating node N2.

As shown in FIG. 6, a second side 407 of the nanopore resistance Rₙₚ is connected to the common electrode 211. The common electrode 211 may be the cis electrode 1103 as shown in FIG. 1A. In some embodiments, during the calibration mode, the voltage applied to the common electrode 211 and the second input terminal 213 of the amplifier 209-1 are set to the same voltage level (e.g., V_{ref}). During the measurement mode, the voltage applied at the common electrode 211 and the second input terminal 213 of the amplifier 209-1 can be different. Detailed descriptions about the operation of the bias compensation circuit 317 will be discussed below.

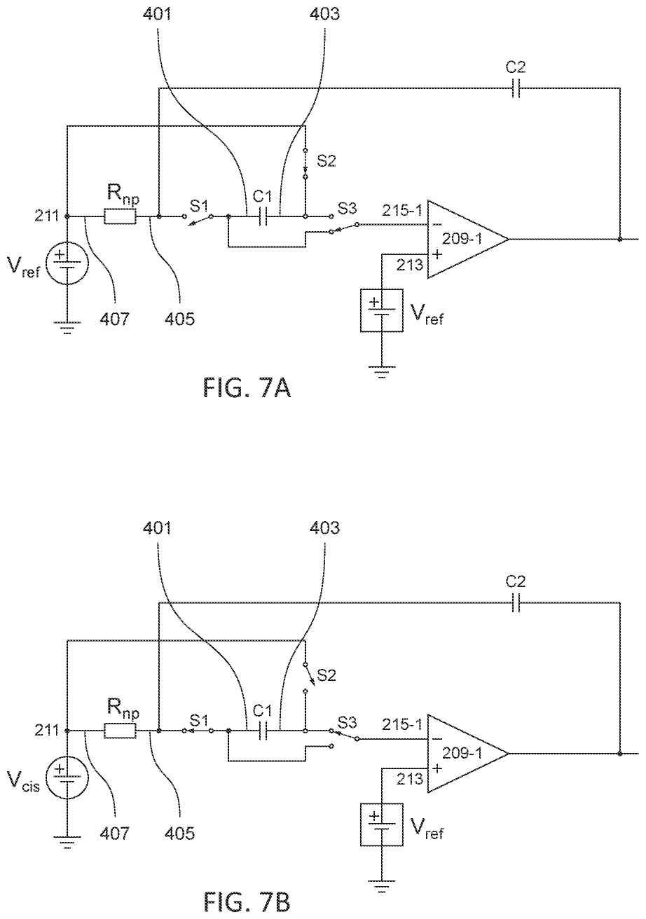

FIG. 7A illustrates an example circuit configuration when the bias compensation circuit 317 of FIG. 6 operates in a calibration mode according to some embodiments of the disclosed technology. As described above, the example circuit configuration of FIG. 7A can be obtained by connecting the first switch S1 to the floating node N1, connecting the second terminal 403 of the offset capacitor C1 to the common electrode 211 through the second switch S2, and connecting the first terminal 401 of the offset capacitor C1 to the first input terminal 215-1 of the amplifier 209-1 through the third switch S3. As illustrated in FIG. 7A, both the voltages at the second input terminal 213 of the amplifier 209-1 and the common electrode 211 are biased at V_{ref}. With the presence of bias offset of the amplifier 209-1, the voltage at the first input terminal 215-1 of the amplifier 209-1 becomes V_{ref}+V_{offset}. During the calibration mode, the offset capacitor C1 is inversely connected. In other words, the second terminal 403 of the offset capacitor C1 is connected to the common electrode 211, which is at a voltage level equaling V_{ref}; and the first terminal 401 of the offset capacitor C1 is connected to the first input terminal 215-1 of the amplifier 209-1, which is at a voltage level equaling V_{ref}+V_{offset}. As such, the offset capacitor C1 is inversely charged to a voltage level that equals V_{ref}−(V_{ref}+V_{offset})=−V_{offset}.

FIG. 7B illustrates an example circuit configuration when the bias compensation circuit 317 of FIG. 6 operates in a measurement mode according to some embodiments of the disclosed technology. As described above, the example circuit configuration of FIG. 7B can be obtained by connecting the first terminal 401 of the offset capacitor C1 to the first side 405 of the nanopore resistance Rₙₚ through the first switch S1, connecting the second terminal 403 of the offset capacitor C1 to the first input terminal 215-1 of the amplifier 209-1 through the third switch S3, and connecting the second switch S2 to the floating node N2. As shown in FIG. 7B, the voltage at the second input terminal 213 of the amplifier 209-1 is still set as V_{ref} but the voltage at the common electrode 211 is now biased at V_{cis}. As such, the voltage at the first input terminal 215-1 of the amplifier 209-1 remains V_{ref}+V_{offset}. The first terminal 401 of the offset capacitor C1 is connected to the first side 405 of the nanopore resistance Rₙₚ and the second terminal 403 of the offset capacitor C1 is connected to the first input terminal 215-1 of the amplifier 209-1. Thus, the voltage level across the nanopore resistance Rₙₚ equals the voltage level at the common electrode 211 (i.e., V_{cis}) minus the voltage across the offset capacitor (i.e., −V_{offset}), and further minus the voltage level at the first input terminal 215-1 (i.e., V_{ref}+V_{offset}) of the amplifier 209-1. In other words, the voltage across the nanopore resistance Rₙₚ equals V_{cis}−(−V_{offset})−(V_{ref}+V_{offset})=V_{cis}−V_{ref}.

As such, the voltage across the nanopore resistance Rₙₚ is independent from the bias offset of the amplifier 209-1. Advantageously, the cancellation of the bias offset may lead to better measurement for nanopore sequence, in contrast to the scenario illustrated in FIG. 3A. The output of the amplifier 209-1 may then be processed using either analog or digital processing techniques to determine the voltage variation of an associated nanopore sensor (e.g., nanopore sensor 201-1 of FIG. 2). The voltage variation of the nanopore sensor may then be used to decide the identity of the polynucleotides that passes through the nanopore sensor. Although not illustrated in FIG. 7B, in some embodiments, the output of the amplifier 209-1 is fed into an analog-to-digital converter (ADC) during the measurement. In other words, the output of the amplifier 209-1 is fed to the ADC for generating a digital signal representative of the output of the amplifier 209-1. As such, the response associated with a polynucleotide passing through a nanopore sensor 201-1 can be evaluated using digital signal processing techniques.

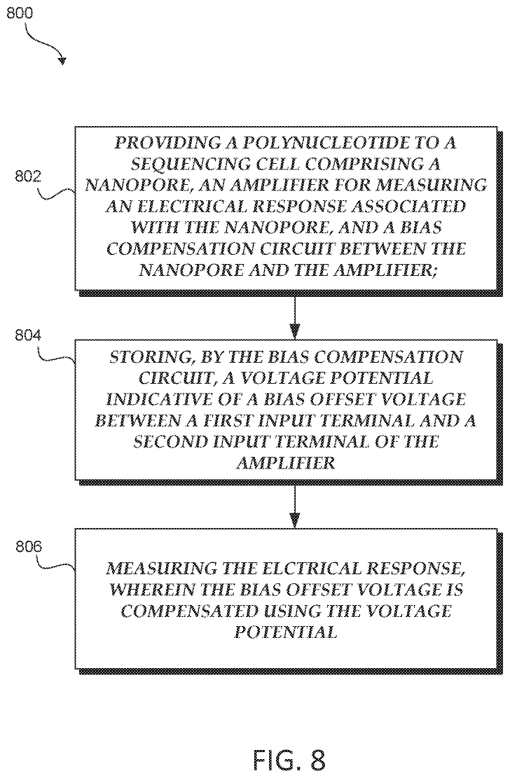

FIG. 8 is a flow diagram depicting a method 800 for compensating bias offsets in the amplifier array of FIG. 2 in accordance with some embodiments of the disclosed technology.

Block 802 includes providing a polynucleotide to a sequencing cell that has a nanopore sensor. The sequencing cell include a nanopore sensor shown in FIG. 2. For example, the sequencing cell may include the nanopore sensor 201-1, the liquid conductor 205 and the metallic pad 207 paired with the nanopore sensor 201-1. Alternatively, the sequencing cell can include other nanopore sensor (e.g., one of the nanopore sensors 201-2 through 201-N) and corresponding liquid conductor 205 and metallic pad 207. As such, block 802 may provide a polynucleotide to the nanopore sensor 201-1, or provide a polynucleotide to nanopore sensor 201-2 or 201-N. Additionally, the sequencing cell can further include an amplifier 209-1 for measuring an electrical response associated with the nanopore sensor 201-1, and a bias compensation circuit between the nanopore sensor 201-1 and the amplifier 209-1.

At block 804, a bias compensation circuit stores a voltage potential that is indicative of an input offset voltage between a first input terminal and a second input terminal of an amplifier 209-1. For example, the bias compensation circuit may store the input offset voltage of the amplifier 209-1.

At block 806, the electrical response of the nanopore sensor is measured, where the bias offset voltage of the amplifier is compensated using the stored voltage potential. For example, the amplifier 209-1 measures the electrical response associated with the sequencing cell that includes the nanopore sensor 201-1 to provide information about the identity of the polynucleotide that is provided with the nanopore sensor 201-1. Although not shown in FIG. 8, in some embodiments, an electrolyte may be provided to a sequencing cell before the polynucleotide is provided to the sequencing cell.

To independently control or address each unit cell (i.e., sequence cell), in some embodiments, each of the nanopore unit cells in a nanopore array may have its own trans electrode but may share a common cis electrode. In some embodiments, each of the nanopore unit cells in a nanopore array may have its own cis electrode but may share a common trans electrode. In some embodiments, each of the nanopore unit cells in a nanopore array may have its own cis electrode and trans electrode. In some embodiments, each of the nanopore unit cells in a nanopore array may share a common cis electrode and a common trans electrode.

The array may have any suitable number of nanopore unit cells. In some instances, the array comprises about 200, about 400, about 600, about 800, about 1000, about 1500, about 2000, about 3000, about 4000, about 5000, about 10000, about 15000, about 20000, about 40000, about 60000, about 80000, about 100000, about 200000, about 400000, about 600000, about 800000, about 1000000, about 10000000 or more nanopore unit cells. In some instances, the array comprises at least 200, at least 400, at least 600, at least 800, at least 1000, at least 1500, at least 2000, at least 3000, at least 4000, at least 5000, at least 10000, at least 15000, at least 20000, at least 40000, at least 60000, at least 80000, at least 100000, at least 200000, at least 400000, at least 600000, at least 800000, at least 1000000 or at least 10000000 nanopore unit cells. In some cases, the array can include individually addressable nanopore unit cells at a density of at least about 500, 600, 700, 800, 900, 1000, 10,000, 100,000 or 1,000,000 unit cells per mm².

### Additional Notes

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

It is to be understood that the ranges provided herein include the stated range and any value or sub-range within the stated range, as if such value or sub-range were explicitly recited. For example, a range from about 2 nm to about 20 nm should be interpreted to include not only the explicitly recited limits of from about 2 nm to about 20 nm, but also to include individual values, such as about 3.5 nm, about 8 nm, about 18.2 nm, etc., and sub-ranges, such as from about 5 nm to about 10 nm, etc. Furthermore, when “about” and/or “substantially” are/is utilized to describe a value, this is meant to encompass minor variations (up to +/−10%) from the stated value.

While several examples have been described in detail, it is to be understood that the disclosed examples may be modified. Therefore, the foregoing description is to be considered non-limiting.

While certain examples have been described, these examples have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure.

Features, materials, characteristics, or groups described in conjunction with a particular aspect, or example are to be understood to be applicable to any other aspect or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing examples. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations, one or more features from a claimed combination can, in some cases, be excised from the combination, and the combination may be claimed as a sub-combination or variation of a sub-combination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some examples, the actual steps taken in the processes illustrated and/or disclosed may differ from those shown in the figures. Depending on the example, certain of the steps described above may be removed or others may be added. Furthermore, the features and attributes of the specific examples disclosed above may be combined in different ways to form additional examples, all of which fall within the scope of the present disclosure. Also, the separation of various system components in the implementations described above should not be understood as requiring such separation in all implementations, and it should be understood that the described components and systems can generally be integrated together in a single product or packaged into multiple products. For example, any of the components for an energy storage system described herein can be provided separately, or integrated together (e.g., packaged together, or attached together) to form an energy storage system.

For purposes of this disclosure, certain aspects, advantages, and novel features are described herein. Not necessarily all such advantages may be achieved in accordance with any particular example. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain examples include, while other examples do not include, certain features, elements, and/or steps. Thus, such conditional language is not generally intended to imply that features, elements, and/or steps are in any way required for one or more examples or that one or more examples necessarily include logic for deciding, with or without user input or prompting, whether these features, elements, and/or steps are included or are to be performed in any particular example.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

Language of degree used herein, such as the terms “approximately,” “about,” “generally,” and “substantially” represent a value, amount, or characteristic close to the stated value, amount, or characteristic that still performs a desired function or achieves a desired result.

The scope of the present disclosure is not intended to be limited by the specific disclosures of preferred examples in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A device for sequencing polynucleotides, comprising:
a nanopore for sensing a polynucleotide;
an amplifier configured to measure an electrical response associated with the nanopore, wherein the amplifier has a bias offset voltage between a first input terminal and a second input terminal; and
a bias compensation circuit coupled to the nanopore and the amplifier, the bias compensation circuit configured to:
store a voltage potential indicative of the bias offset voltage; and
compensate the bias offset voltage using the voltage potential.

2. The device of claim 1, wherein the bias compensation circuit comprises an offset capacitor, and wherein the offset capacitor stores the voltage potential indicative of the bias offset voltage during a first operation mode.

3. The device of claim 2, wherein the nanopore has a first side and a second side, and wherein the offset capacitor has a first terminal and a second terminal, and wherein during the first operation mode:
the first terminal is operably connected to the first input terminal through a switching circuit; and
the second terminal is operably connected to a common electrode through the switching circuit.

4. The device of claim 3, wherein the common electrode is tied to a ground potential during the first operation mode.

5. The device of claim 3, wherein the offset capacitor is configured to compensate the bias offset voltage using the voltage potential during a second operation mode, and wherein the amplifier measures the electrical response associated with the nanopore during the second operation mode.

6. The device of claim 5, wherein the first terminal is operably connected to the first side of the nanopore and the second terminal is operably connected to the first input terminal through the switching circuit during the second operation mode.

7. The device of claim 1, wherein the nanopore is a polypeptide nanopore or a solid-state nanopore, and wherein the polypeptide nanopore forms an opening in a lipid, polymer, or solid-state membrane.

8. The device of claim 1, wherein the electrical response is an ionic current through the nanopore, and wherein the ionic current is modulated by: nucleotides in the polynucleotide near a sensing zone of the nanopore, labels on nucleotides in the polynucleotide near the sensing zone of the nanopore, nucleotides being incorporated to the polynucleotide, labels on nucleotides being incorporated to the polynucleotide, or any combination thereof.

9. The device of claim 1, further comprising an analog-to-digital-converter, wherein an output of the amplifier is fed to the analog-to-digital converter for generating a digital signal representative of the output of the amplifier.

10. A method for sequencing polynucleotides, comprising:
providing a polynucleotide to a sequencing cell comprising a nanopore, an amplifier for measuring an electrical response associated with the nanopore, and a bias compensation circuit between the nanopore and the amplifier;
storing, by the bias compensation circuit, a voltage potential indicative of a bias offset voltage between a first input terminal and a second input terminal of the amplifier; and
measuring the electrical response, wherein the bias offset voltage is compensated using the voltage potential.

11. The method of claim 10, wherein the voltage potential is stored in an offset capacitor of the bias compensation circuit.

12. The method of claim 11, wherein storing the voltage potential comprises:
connecting a first terminal of the offset capacitor to the first input terminal of the amplifier through a switching circuit; and
connecting a second terminal of the offset capacitor to a common electrode through the switching circuit.

13. The method of claim 12, further comprising setting the common electrode and the second input terminal of the amplifier to a ground potential and thereby inversely charging the offset capacitor to the voltage potential equal to a voltage at the first input terminal of the amplifier.

14. The method of claim 12, further comprising setting the common electrode and the second input terminal of the amplifier to a reference potential and inversely charging the offset capacitor to the voltage potential equal to a difference between voltages at the first input terminal and the second input terminal of the amplifier.

15. The method of claim 11, wherein measuring the electrical response comprises:
connecting a first terminal of the offset capacitor to a first side of the nanopore through a switching circuit, while a second side of the nanopore is connected to a common electrode; and
connecting a second terminal of the offset capacitor to the first input terminal of the amplifier through the switching circuit.

16. The method of claim 15, further comprising setting the common electrode to a ground potential and processing an output of the amplifier, wherein the output of the amplifier is indicative of the electrical response.

17. The method of claim 15, further comprising setting the common electrode to a biased potential and processing an output of the amplifier, wherein the output of the amplifier is indicative of the electrical response.

18. The method of claim 10, further comprising converting an output of the amplifier to a digital signal using an analog-to-digital converter.

19. The method of claim 10, wherein the electrical response measured by the amplifier is an ionic current through the nanopore or equivalents thereof.

20. The method of any of claim 10, further comprising providing an electrolyte to the sequencing cell prior to providing the polynucleotide.

21. A system for sequencing polynucleotides, comprising:
a common cis well associated with a common cis electrode; and
a plurality of sequencing cells, each of the plurality of sequencing cells comprising:
a trans well associated with a trans electrode;
a nanopore for sensing a polynucleotide, the nanopore fluidically connecting the trans well to the common cis well;
an amplifier configured to measure an electrical response in the nanopore, the amplifier having a bias offset voltage between a first input terminal and a second input terminal; and
a bias compensation circuit coupled to the nanopore and the amplifier, the bias compensation circuit configured to:
store a voltage potential indicative of the bias offset voltage; and
compensate the bias offset voltage using the voltage potential.

22. The system of claim 21, wherein the bias compensation circuit comprises an offset capacitor, and wherein the offset capacitor stores the voltage potential indicative of the bias offset voltage during a first operation mode.

23. The system of claim 22, wherein the offset capacitor compensates the bias offset voltage of the amplifier during a second operation mode, and wherein the amplifier measures the electrical response in the nanopore during the second operation mode.

24. The system of claim 21, wherein the plurality of sequencing cells form a two-dimensional (2D) array.

25. The system of claim 24, wherein a sequencing cell density of the 2D array is at least 500 sequencing cells per mm².

26. The system of claim 24, wherein the 2D array includes at least 1000 sequencing cells.
